(19) United States
(12) Patent Application Publication
(10) Pub. No.: US 2013/0289670 A1
(43) Pub. Date: Oct. 31, 2013

(54) DEVICE FOR MEDICAL EXTERNAL TREATMENT BY LIGHT

(71) Applicants: Rolf Thiberg, Akersberga (SE); Per Olausson Lido, Tyreso (SE)

(72) Inventors: Rolf Thiberg, Akersberga (SE); Per Olausson Lido, Tyreso (SE)

(73) Assignee: BIOLIGHT PATENT HOLDING AB, Stockholm (SE)

(21) Appl. No.: 13/989,898

(22) PCT Filed: Nov. 14, 2011
(86) PCT No.: PCT/SE2011/051363

§ 371 Date: Jul. 11, 2013

(30) Foreign Application Priority Data
Dec. 3, 2010 (SE) 1051282-0
Dec. 3, 2010 (SE) 1051283-8

(51) Int. Cl. A61N 5/06

(52) U.S. Cl. CPC A61N 5/06
USPC 607/88

(57) ABSTRACT

A device for external medical treatment with light for treating and/or curing different illnesses includes a light emitting unit, arranged to abut against or be held near the body of an individual, and a drive device for the light emitting unit. The unit includes light emitting diodes or the corresponding, which light emitting diodes are arranged to emit light of different wavelength intervals. The drive device is arranged to control the light emitting unit to emit one or several lights during one or several predetermined times and to, in a pulsating manner, emit the light according to a predetermined pulse frequencies or series of pulse frequencies during such times. The light emitting unit includes a cover and a surface carrying the light emitting diodes, and where the light emitting diodes are positioned on the plate. The diode surface has a curved shape adapted to the body part to be treated.

The present invention relates to a device for external medical treatment with light, for alleviating and/or curing different medical conditions.

In for example the Swedish patent no 502 784, a device is described for external medical treatment with light, comprising a light emitting means arranged to abut against or be held near the body of an individual, as well as a drive device for the light emitting means, which light emitting means comprises light emitting diodes or the corresponding, and is arranged to emit infrared light. The invention according to the said patent specifies that the drive device is arranged to control the light emitting means to, in a first step, emit infrared light during a first predetermined time, and to thereafter, in a second step, emit visible light during a second predetermined time, and that the drive device is arranged to control the light emitting means to emit the infrared light and the visible light, respectively, in a pulsating manner according to a predetermined series of pulse frequencies.

It is also known to emit other monochromatic lights for treatment of various medical conditions.

Furthermore, it has turned out that treatment with only one or several monochromatic lights and other than infrared lights, such as visible light of various colours, which is emitted according to a certain pulse frequency, results in very good treatment results.

It has turned out that a device of the said type very successfully can be used for treatment of many medical conditions and injuries, for example athletic injuries, strains, muscular pain, joint pains, headache, various inflammatory conditions, various skin disorders such as acne, back pain, etc., under the condition that the lights are emitted in a certain way. Hereby, the light treatment has a positive effect on the healing process for injuries, and it alleviates and/or cures various illnesses.

The device has also very successfully been used for treatment of wounds, for example of diabetic foot wounds and within geriatric care, and also within aesthetics and equestrian sports.

Hence, there is a knowledge that light treatment through the emission of a certain light in certain frequency series results in a substantially increased effect in the form of shortening of the time for the curing or alleviation of an illness.

Since the light treatment is presumed to have effect already at the cell level, it is desirable to use a dynamic light image and that the whole area on the body to be treated receives an equal amount of light over time.

A problem with the mentioned devices, for example those described in the Swedish patent no. 502 784 above, is that the treating personnel, in order to fulfil the requirements of a dynamic light image and an amount of light which is as equally distributed as possible over time, must perform an oscillating motion with the light emitting means when it abuts against or is held near the area on the body of the individual to be treated. The reason for this is that the light emitting diodes that are arranged at the bottom of the light emitting means have a certain geometric extension, and are of different types, why there is a certain distance between two adjacent light emitting diodes of the same type. Therefore, in order for the whole area to be treated to be exposed to an illumination which is as even as possible, the light emitting means must be brought back and forth across the area.

Since a treatment typically lasts about two to ten minutes, it is very burdensome for the personnel to perform such a treatment often.

Therefore, it is already known to arrange an eccentric device between the cover of the light emitting means and the plate carrying the light emitting diodes, see Swedish patent no 515 992. In this case, the eccentric device comprises a first part which is fixed relative to the cover, a second part which is connected to the light emitting diode plate and a spring arranged to prevent rotation of the plate. An electric motor is arranged to drive the eccentric device, whereby the plate with the light emitting diodes performs an oscillating motion.

It is also already known to arrange an electric motor fixed relative to the cover of the light emitting means, which is connected to the light emitting diode plate via a drive axis, see Swedish patent no 515 991. Hereby, the plate with the light emitting diodes will perform a rotational motion, whereby a smoother illumination is achieved in total, and whereby increased cell response is achieved in the area to be treated.

A disadvantage with the above described devices for achieving the required oscillating or rotational motion is that they both build upon a mechanically generated motional pattern, in which expensive and relatively heavy components are built into the device. In turn, this leads to higher production costs, increased wear and thereby increased cost for maintenance and risk for complaints. Desires for as friction-free components as possible result in tight tolerances during manufacture and harsh demands during assembly of the moving parts.

A further disadvantage with today's equipment for creating an even illumination is that the mechanically generated motional pattern is conducted in a plane, and therefore limits the geometry of the emitting diode plate to such a plane. If the illuminated surface is also planar, this is not a problem, since an even and sufficient illumination is achieved across the surface. However, in reality the surface to be treated is seldom planar. Therefore, there is a desire to increase the degree of freedom with respect to the geometric shape of the emitting diode plate, so that it becomes more adapted to the body part which is to be treated.

Hence, the present invention relates to a device for external medical treatment with light, comprising a light emitting means arranged to abut against or be held near the body of an individual, as well as a drive device for the light emitting means, which light emitting means comprises light emitting diodes or the corresponding, which light emitting diodes are arranged to emit light of different wavelength intervals, wherein the drive device is arranged to control the light emitting means to emit one or several lights during one or several predetermined times and to, in a pulsating manner, emit the said light according to a predetermined pulse frequency or series of pulse frequencies during said times, which light emitting means comprises a cover and a light emitting diode plate, carrying the light emitting diodes, and where the light emitting diodes are positioned on said plate so that there is a certain distance between two adjacent light emitting diodes, and is characterised in that an electric drive circuit is arranged for controlling the light emitting diodes, which drive circuit is connected to one or several of the individual light emitting diodes and arranged to switch them on and off so that a movable light image appears across the surface of the light emitting diode plate.

According to a preferred embodiment of the present invention, the said problem with a non planar surface to be treated is solved by giving the emitting diode surface a curved shape, and by replacing the mechanical rotation with an electronically generated motional pattern. An electronically generated motional pattern gives a substantially larger freedom with respect to the desired motion along the body part to be treated, and does not comprise any mechanically movable parts. This gives a possibility to design the emitting diode plate in a shape which is suited to the body part to be treated. It also gives a possibility to a more random motion along the body part to be treated.

Hereby, the emitting diode surface has a curved shape for adaptation towards the body part to be treated, and an electric drive circuit is arranged for sequential or random control of the light emitting diodes, which drive circuit is connected to one or several of the individual light emitting diodes and arranged to switch them on and off so that a movable light image appears across the curved light emitting diode surface.

According to an advantageous embodiment, the light emitting diodes are arranged in the shape of a matrix in rows and columns on the plate.

In the following, an embodiment of the invention is described in more detail, with reference to the enclosed drawings, whereby

FIG. 1 shows a light emitting means according to the invention in a side view,

FIG. 2 schematically shows a block diagram of a device according to the invention,

FIG. 3 is an explanatory sketch of how different motional patterns can be generated across the surface of the light emitting diode matrix by sequential control of the light emitting diodes with row and column circuits,

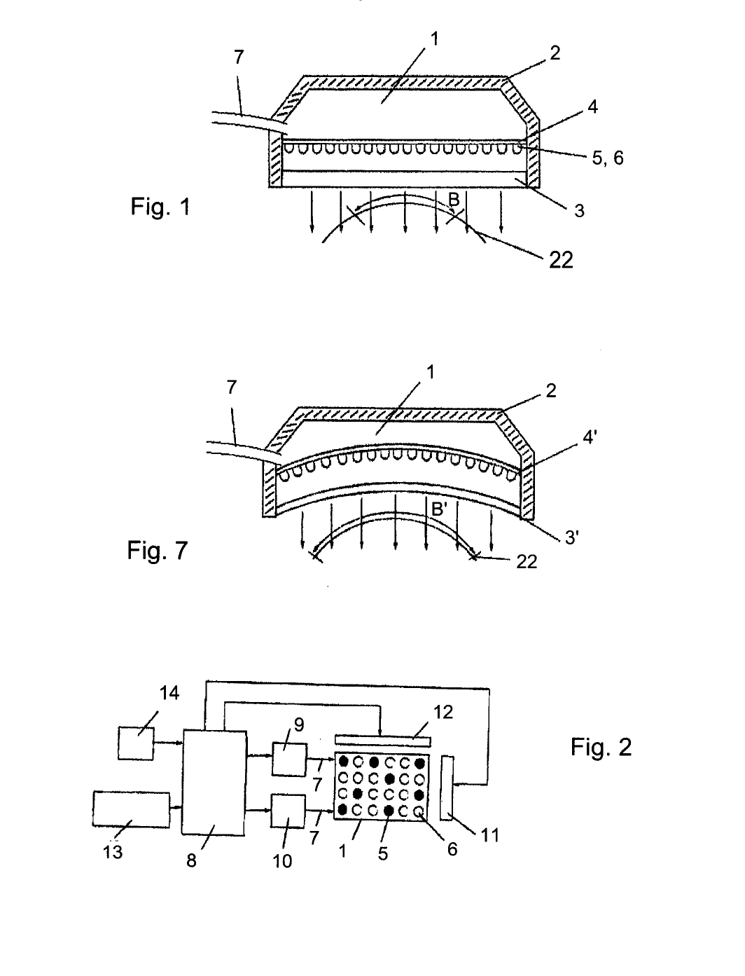

FIG. 4 shows two examples of motional patterns, where several movements are created simultaneously, partly non-overlapping movements and partly overlapping movements,

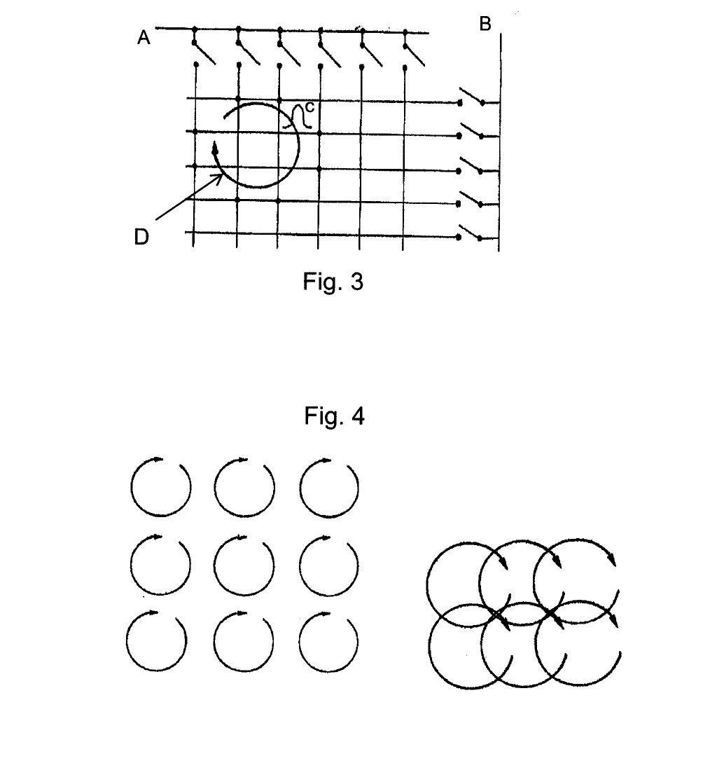

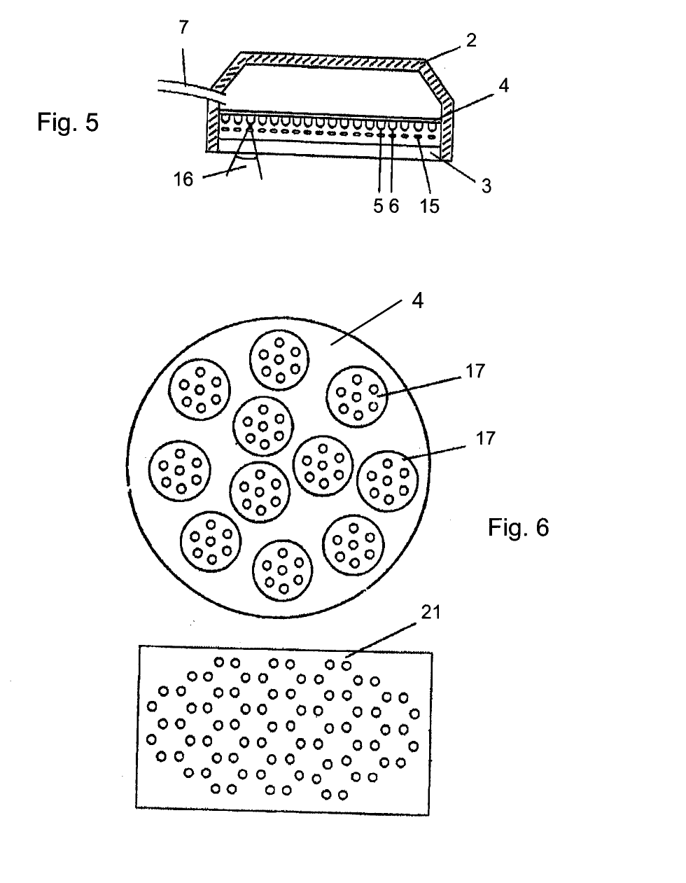

FIG. 5 shows an embodiment wherein the surface mounted light emitting diodes are arranged with optical lenses in order to give a desired light image, and

FIG. 6 shows two exemplary embodiments wherein the light emitting diodes are arranged in clusters on the light emitting means.

FIG. 7 shows a light emitting means according to an embodiment of the invention in a side view,

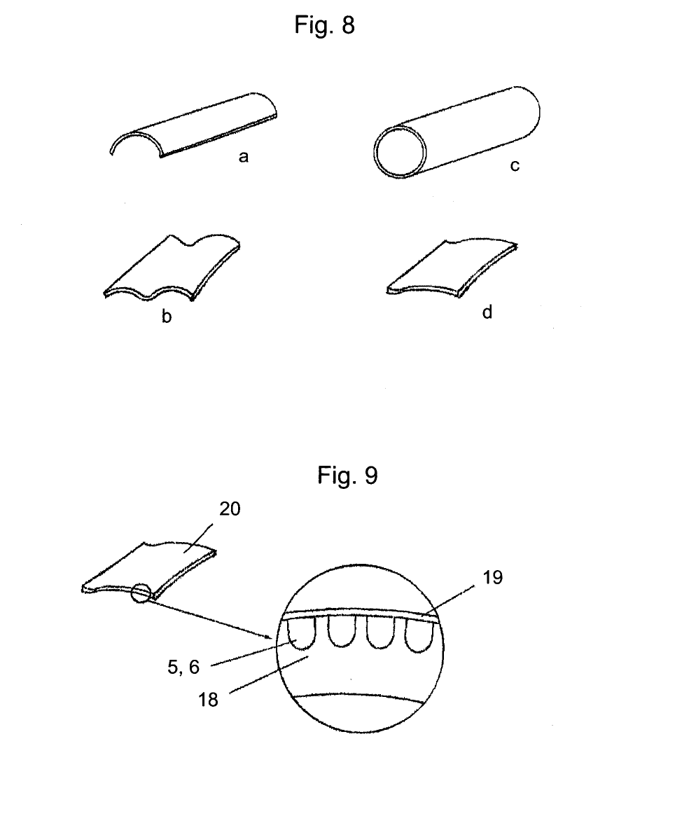

FIG. 8 schematically shows some different examples of suitably designed diode surfaces

FIG. 9 shows an example of how the surface mounted light emitting diodes can be applied.

FIG. 1 shows, generally, a device for external medical treatment with light, comprising a light emitting means 1 arranged to abut against or be held close to the body of an individual, for example an arm or a leg 22. The light emitting means is shown in the figure from the side, and comprises a housing 2 arranged with a transparent pane 3. Under this pane 3, inside the housing, there is a plate 4 in which a number of light emitting diodes 5,6 are arranged in the shape of a matrix. The light emitting diodes 5,6 are arranged to emit light through the pane 3 when supplied with current via a cable 7. During use, the housing 2 is held so that the pane 3 abuts against or is close to the body part in question of for example a person the wound injury of which is to be treated. Furthermore, the device comprises a drive device for the light emitting means, see the block scheme in FIG. 2, wherein the light emitting means 1 is also shown from below. The drive device is arranged to control the light emitting means 1 to emit different monochromatic light of different wavelengths during different predetermined times, and to emit the said light in a pulsating manner according to a predetermined pulse frequency or series of pulse frequencies during the said times.

The light emitting means 1 comprises light emitting diodes 5, 6, arranged to emit a substantially monochromatic, visible light in any of the colours violet, blue, yellow, orange, red or green, as well as infrared and other non-visible wavelengths. Which of these lights that is to be used depends on the illness or type of injury to be treated.

The light emitting means 1 may comprise a type of light emitting diodes 5 which are arranged to emit for instance infrared light. These are marked using filled circles in FIG. 2. Visible light may be emitted using other light emitting diodes 6, which are marked using unfilled circles in FIG. 2. The light emitting diodes 5,6 for infrared light and visible light, respectively, are preferably semiconductors of the type GaAs (Gallium Arsenide). The light emitting diodes can also be arranged to emit light of other wavelengths. Thus, the light emitting means 1 may comprise light emitting diodes 5, 6 arranged to emit an essentially monochromatic, visible light in any of the colours violet, blue, yellow, orange, red or green, as well as infrared and other non-visible wavelengths. Which of these lights that are to be used depends on the illness or type of injury to be treated. The light emitting diodes may be arranged in the shape of a matrix, in rows and columns, on the light emitting means 1.

The drive device for the light emitting means comprises, in a way which is known as such, a computer 8 for controlling drive circuits 9,10,11,12, which drive circuits are fed with signals from the computer and in turn drive the light emitting diodes via the conductor 7. To the drive device or the computer, a keyboard 13 is connected, using which the operator can key in data in order to thereby control the drive device to control the light emitting means in a desired way. Suitably, there is also a display 14 for showing the settings input via the keyboard 13. Furthermore, the device comprises an electric drive circuit, arranged for sequential or otherwise predetermined control of the light emitting diodes, which drive circuit is connected to the individual light emitting diodes and is arranged to switch on and off the light emitting diodes, for example in sequence so that a movable light image appears across the surface of the light emitting diode matrix.

According to a first preferred embodiment, the drive circuit 9,10,11,12 is arranged for arbitrary control of the light emitting diodes, so that they are switched on and off arbitrarily.

According to a second preferred embodiment, the drive circuit 9,10,11,12 is arranged for stochastic control of the light emitting diodes, so that they are switched on and off stochastically.

According to a third preferred embodiment, the drive circuit 9,10,11,12 is arranged for a predetermined control of the light emitting diodes, so that they are switched on and off according to a predetermined pattern.

In FIG. 2, the said drive circuit is shown schematically and connected to the rows and columns of the diode matrix. According to an advantageous embodiment, the drive circuit comprises a separate drive circuit 11, arranged to control one or several rows of light emitting diodes in the matrix, and a separate drive circuit 12, for controlling one or several columns of light emitting diodes in the matrix. The drive circuits 11,12 are connected to the computer 8 and a suitable power source (not shown). The processor of the computer can control, in a standard way, the drive circuits for rows and columns in the diode matrix so that a suitable movable light image appears across the surface of the diode matrix. By switching on and off the individual light emitting diodes in sequence, the light intensity moves across the surface. A light pulse/light image thereby appears and an arbitrary movement, for example a circle motion, can be generated, independent of if the diode surface is planar or curved. This movement replaces the earlier mechanically generated movement of the light emitting means, which for mechanical reasons was limited to planar surfaces.

How different motion patterns can be generated across the surface of the light emitting diode matrix through sequential control of the light emitting diodes with row- and column drive circuits is principally shown in FIG. 3. In the figure, a matrix schema is shown, with a thought light emitting diode in each junction and a switch for columns (A) and a switch for rows (B).

An arbitrarily selected light emitting diode can be switched on by closing one switch (A) and one switch (B). By replacing the switches with transistors or drive circuits according to the invention, the current can also be varied, and thereby the light intensity in an arbitrarily selected light emitting diode. By ramping of the light from the light emitting diodes, in other words by increasing the light intensity of a diode at the same time as the light intensity is reduced for a nearby positioned diode, one or several wandering light pulses (C) of suitable shape may be generated. By ramping of the light from the light emitting diodes, a desired dynamic light image may be achieved, and a smoother light intensity distribution across the matrix surface. Ramping can take place analogously or using so called pulse width modulation (PWM).

By switching individual light emitting diodes on and off, an arbitrary movement can be achieved, for example a circular motion, as is indicated in FIG. 3. The light pulse C “wanders” along the circle path D as indicated in the figure. Several movements can also be generated simultaneously, see FIG. 4, and overlapping motions may also be generated if desired, see FIG. 4. The type of movement pattern to be used depends on which type of injury to be treated, and is controlled using a suitable software in the computer 8. It is realised that the electronically generated movement admits a freer choice of movement patterns as compared to mechanically generated movements.

As is clear from FIG. 1, the light emitting diode surface 4 in which the light emitting diodes 5,6 are mounted is mainly planar. This is a legacy from the previously mechanically generated movement pattern. As is clear from the figure, then the lighting is only optimal on a smaller part of the body part in question, indicated by arrow B. This results in the equipment often having to be moved around the body part.

By generating the diode movement electronically, it is possible to design the light diode surface 4 in a curved manner, as shown in FIG. 7. This way, the light emitting diode surface can be more adapted to the body part to be treated. During use, the housing 2 is held so that the curved pane 3′ abuts against or is held close to the body part in question. Then, a larger part of the body part can be illuminated, as is indicated by arrow B′, see FIG. 7.

FIG. 8 schematically shows a few examples of suitable surface geometries adapted to treating different body parts: a surface curved in one dimension in FIG. 8a, a surface curved in two dimensions in FIG. 8b, a tube-shaped surface in FIG. 8c, and a flexible, body shaped surface in FIG. 8d. The surfaces may be built up from a preformed, rigid material, but they may also advantageously be built up in a flexible material. In the latter case, the illumination component can be in contact with the body part in question, and the shape of the diode surface is then determined by the outer shape of the body part. In the case of a preformed, rigid diode surface, this is held at a suitable distance from the body part during treatment.

FIG. 9 shows an example of how the surface mounted light emitting diodes 5, 6 can be applied in a flexible “light mat” 20 with a shape which is adapted to the current body part. The material is for example a soft plastic material with a certain “stretch”. The light emitting diodes can be arranged with built-in optical lenses to give a desired light image for the individual light emitting diodes as compared to the spread angle they have as such. The light emitting diodes with optics are embedded in a translucent material 18, a soft plastic material, such as polyethylene or the like, functioning as distance element so that a suitable distance between the light emitting diodes/optics and current body part is maintained. Thus, a light emitting diode mat 20 can simply be applied onto or be clamped to the body. The light emitting diode mat is arranged to completely or partly surround the current body part. The light emitting diodes are connected to the drive circuits via a flexible PCB (Printed Circuit Board), a so called flexiboard 19.

According to a preferred embodiment, the diode surface 4′ is built up from a flexible material for abutment against the body part 22 to be treated, whereby the shape of the diode surface is determined by the outer shape of the body part. Please see FIG. 7.

According to an alternative embodiment, the diode surface 4′ is preshaped in a rigid material. Please see FIG. 7. According to a preferred embodiment, lenses 15 are integrated in a plate which is mounted over the light emitting diodes. Please see FIG. 5.

FIG. 5 shows an embodiment in which the surface mounted light emitting diodes 5, 6 are provided with optical lenses 15 for providing a desired light image, preferably a decreased light scattering for the individual light emitting diodes as compared to the spread angle they have as such and as indicated by 16 in FIG. 5. In the example shown in the figure, it is intended that small lenses 15 are separately mounted over each light emitting diode. Alternatively, a plate with several lenses in the same body may be mounted over the light emitting diodes.

As an alternative to the matrix with rows and columns of light emitting diodes, the light emitting diodes may be arranged in clusters on the plate 4, as is shown in a few examples in FIG. 6. In this case, the light emitting diodes form round “islands”, clusters 17,21, which are individually controlled so that the clusters for example give a rotating light image.

Thanks to the increased degree of freedom with respect to the shape of the diode plate, treatment equipment can be supplied with several different lighting means for different applications. For instance, the equipment may comprise a number of fixed, predetermined shapes in case no physical contact with the body is desired during the treatment operation, and one or several flexible diode mats, of a sock-type, girdle-type, armlet-type, bandage-type, or quilt-type model, in case the body part in question can determine the shape through contact.

Using the invention, in which the light emitting diodes are switched off and on according to a certain pattern, the above mentioned rotational movement, as well as the above mentioned oscillating movement, can be achieved without the light emitting diodes of the light emitting means moving relative to the body part to be treated. Other movements can of course also be achieved only by switching off and on the light emitting diodes in a predetermined way.

When the light emitting diode surface is shaped after the body part to be treated, a smooth illumination is furthermore achieved across the whole of the said body part.

The invention is not limited to the above described embodiments, but may be varied within the scope of the subsequent claims. Thus, the invention is not limited to the diode shapes described above, but may be applied to any preshaped or flexible bandages and armlets and the like for external medical treatment. Nor is the invention limited to any special layout or any specific pattern of movement for the surface mounted light emitting diodes. It is furthermore realised that the light emitting diodes may be distributed across the entire curved diode surface or only across a part of the same.

1-18. (canceled)

19. Device for external medical treatment with light, comprising a light emitting means (1) arranged to abut against or be held near the body of an individual, as well as a drive device (9,10,11,12) for the light emitting means, which light emitting means comprises light emitting diodes or the corresponding (5,6), which light emitting diodes are arranged to emit light of different wavelength intervals, wherein the drive device (9,10,11,12) is arranged to control the light emitting means (1) to emit one or several lights during one or several predetermined times and to, in a pulsating manner, emit the said light according to a predetermined pulse frequency or series of pulse frequencies during said times, which light emitting means comprises a cover (2) and a light emitting diode plate (4), carrying the light emitting diodes, and where the light emitting diodes are positioned on said plate so that there is a certain distance between two adjacent light emitting diodes, and where an electric drive circuit (9,10,11,12) is arranged for controlling the light emitting diodes, which drive circuit is connected to one or several of the individual light emitting diodes (5,6), characterized in that said drive circuit is arranged to switch the light emitting diodes on and off so that a movable light image appears across the surface of the light emitting diode plate (4) to obtain a movable pattern for light treatment.

20. Device according to claim 19, characterized in that the diode surface (4′) has a curved shape for adaptation to the body part (8) to be treated.

21. Device according to claim 19, characterized in that the drive circuit (9,10,11,12) is arranged for sequential control of the light emitting diodes so that they are switched on and off in sequence.

22. Device according to claim 19, characterized in that the drive circuit (9,10,11,12) is arranged for an arbitrary control of the light emitting diodes so that they are switched on and off arbitrarily.

23. Device according to claim 19, characterized in that the drive circuit (9,10,11,12) is arranged for a stochastic control of the light emitting diodes so that they are switched on and off stochastically.

24. Device according to claim 19, characterized in that the drive circuit (9,10,11,12) is arranged for a predetermined control of the light emitting diodes so that they are switched on and off according to a predetermined pattern.

25. Device according to claim 19, characterized in that the light emitting diodes (5,6) are arranged in a matrix shape in rows and columns on said plate (4).

26. Device according to claim 25, characterized in that the drive circuit comprises a separate drive circuit (11), arranged to control one or several rows of light emitting diodes in the matrix, and a separate drive circuit (12) for controlling one or several columns of light emitting diodes in the matrix.

27. Device according to claim 19, characterized in that the light emitting diodes (5,6) are arranged in clusters on said plate (4), whereby the drive circuit is arranged for sequential control of the diodes in clusters.

28. Device according to claim 19, characterized in that the drive circuits (9,10,11,12) are arranged to increase the light intensity for a diode at the same time as the light intensity is reduced for a nearby located diode, so that one or several moving light pulses (C) of suitable shape are generated.

29. Device according to claim 28, characterized in that the light intensity is arranged to be increased or reduced, respectively, by ramping (toning up and toning down), whereby a generally even illumination of the area to be treated is achieved.

30. Device according to claim 19, characterized in that the surface mounted light emitting diodes (5,6) are provided with optical lenses (15) to give a desired light image for the individual light emitting diodes as compared to the spread angle they have as such.

31. Device according to claim 30, characterized in that the lenses (15) are integrated in a plate which is mounted over the light emitting diodes.

32. Device according to claim 20, characterized in that the diode surface (4′) is preshaped in a rigid material.

33. Device according to claim 20, characterized in that the diode surface (4′) is built up in a flexible material for abutment against the body part (8) to be treated, whereby the shape of the diode surface is determined by the outer shape of the body part.

34. Device according to claim 33, characterized in that the light emitting diodes (5,6) are surface mounted and arranged in a flexible light emitting diode mat (17;20), which completely or partly is arranged to surround the current body part.

35. Device according to claim 19, characterized in that the light emitting diodes with optics are enclosed in a translucent material (18), which functions as a distance element so that a suitable distance between the light emitting diodes/optics and the current body part is maintained.

36. Device according to claim 19, characterized in that the light emitting diodes are connected to the drive circuit via a flexible PCB, a flexible circuit board or the like, a so called flexiboard (19).
